(19) United States
(12) Patent Application Publication
(10) Pub. No.: US 2004/0214889 A1
(43) Pub. Date: Oct. 28, 2004

(54) Calcilytic compounds

(75) Inventors: Maxine Gowen, King of Prussia, PA (US); Larry J. Suva, Little Rock, AR (US); John Fox, Salt Lake City, UT (US); George B. Stroup, King of Prussia, PA (US); Edward F. Nemeth, Toronto (CA)

Correspondence Address: GLAXOSMITHKLINE, Corporate Intellectual Property - UW2220, P.O. Box 1539, King of Prussia, PA, 19406-0939, US

(73) Assignees: SmithKline Beecham Corporation; NPS Pharmaceuticals, Inc.

(21) Appl. No.: 10/852,557

(22) Filed: May 24, 2004

Related U.S. Application Data
(63) Continuation of application No. 10/049,348, filed on Jan. 30, 2002, now abandoned.
Application No. 10/049,348 is a 371 of international application No. PCT/US00/20834, filed on Jul. 31, 2000.
(60) Provisional application No. 60/146,778, filed on Jul. 31, 1999.

(51) Int. Cl. A61K 31/277

(52) U.S. Cl. 514/523

(57) ABSTRACT

Novel methods of treating bone diseases or disorders are provided.

### FIELD OF INVENTION

The present invention relates to the treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease and osteoporosis. The present methods involve the co-administration of an orally active antagonist of the calcium receptor with an anti-resorptive agent.

In mammals, extracellular Ca²⁺ is under tight homeostatic control and regulates various processes such as blood clotting, nerve and muscle excitability, and cellular function. Extracellular Ca²⁺ inhibits the secretion of parathyroid hormone (“PTH”) from parathyroid cells, inhibits bone resorption by osteoclasts, and stimulates secretion of calcitonin from thyroid C-cells. Calcium receptor proteins enable certain specialized cells to respond quickly to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on osteoclasts has also been studied. See Zaidi, Bioscience Reports 10:493, 1990.

Various compounds are known to mimic the effects of extra-cellular Ca²⁺ on calcium receptors. Calcilytics are compounds able to antagonize calcium receptor activity, thereby causing a decrease in one or more calcium receptor activities evoked by extracellular Ca²⁺. Calcilytics are useful as lead molecules in the discovery, development, design, modification and/or construction of calcium receptor modulators which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral metabolisim.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease and osteoporosis.

It is well known that chronic elevation of PTH, such as that seen in hyperparathyroidism, leads to osteoclast-mediated bone loss and abnormal bone histology. Dobnig and Turner, Endocrinol., Vol. 138, pp. 4607-4612 (1997), showed that subcutaneous infusion of high doses of PTH (40 and 80 ug/kg/day) over periods of 2 hours or more led to rapid loss in body weight, hypercalcemia and histological abnormalities in the skeleton consistent with changes seen in hyperparathyroidism. The literature indicates that, while intermittent administration of PTH is desirable in effecting bone formation, if the PTH elevation is too prolonged, bone resorption is elevated. This limitation in the duration of PTH elevation limits the choice of compounds that could be used to antagonize the calcium receptor.

Therefore, there exists a need in the industry for a therapy that could utilize calcium receptor antagonists which might elicit transient PTH elevation without the concomitant resorption problems evidenced in the literature.

There is a further need for a therapy that causes relatively low degrees of PTH elevation, while having the same beneficial effects as the treatments currently available.

### SUMMARY OF THE INVENTION

The present invention provides novel methods of treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease and osteoporosis.

The present methods involve the co-administration of a calcilytic agent with an anti-resorptive agent to a patient in need of treatment. The present calcilytic agents include agents which may cause prolonged PTH elevation. Preferably, the present agents cause a transient elevation of PTH.

### DETAILED DESCRIPTION OF DRAWING

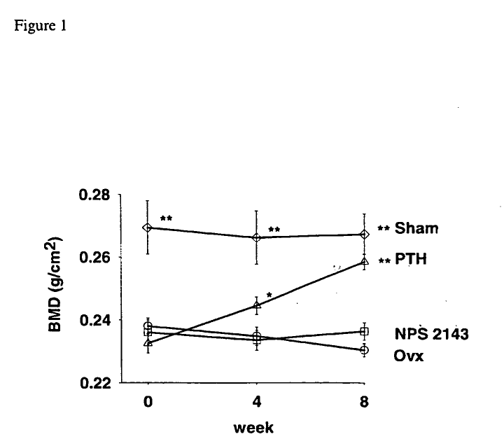

FIG. 1 represents proximal tibial BMD in osteopenic rats following treatment with calcilytic or PTH according to Study 1.

Seven month old rats were ovx and allowed to develop osteopenia for two months. Sham operated rats were treated with vehicle (⋄), ovx rats were treated with vehicle (∘), NPS 2143 100 umol/kg p.o. (□), or rat PTH 1-34 5 ug/kg s.c. (Δ). BMD was measured at the time points indicated. Statistical significance is indicated: * P<0.05; **P<0.01

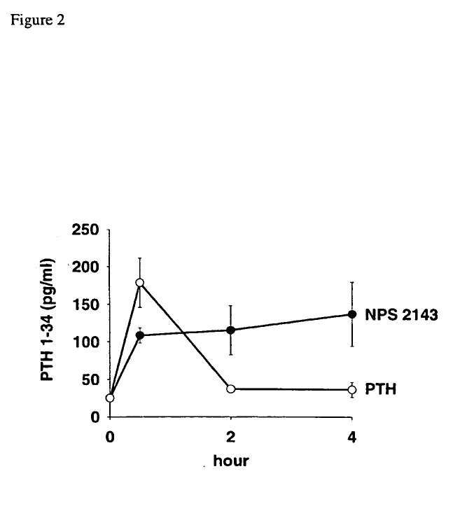

FIG. 2 represents plasma PTH levels in osteopenic rats treated with calcilytic or rat PTH according to Study 1.

Timed plasma samples were collected relative to administration of the agent as indicated, following treatment with calcilytic adminstered (filled circle) or PTH (open circle).

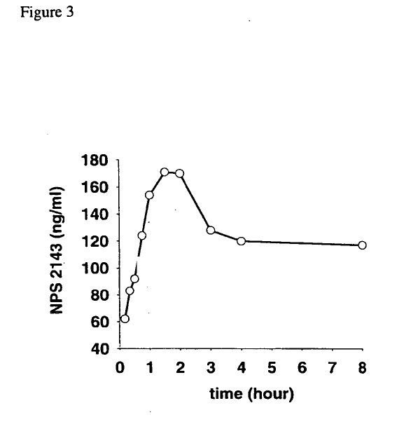

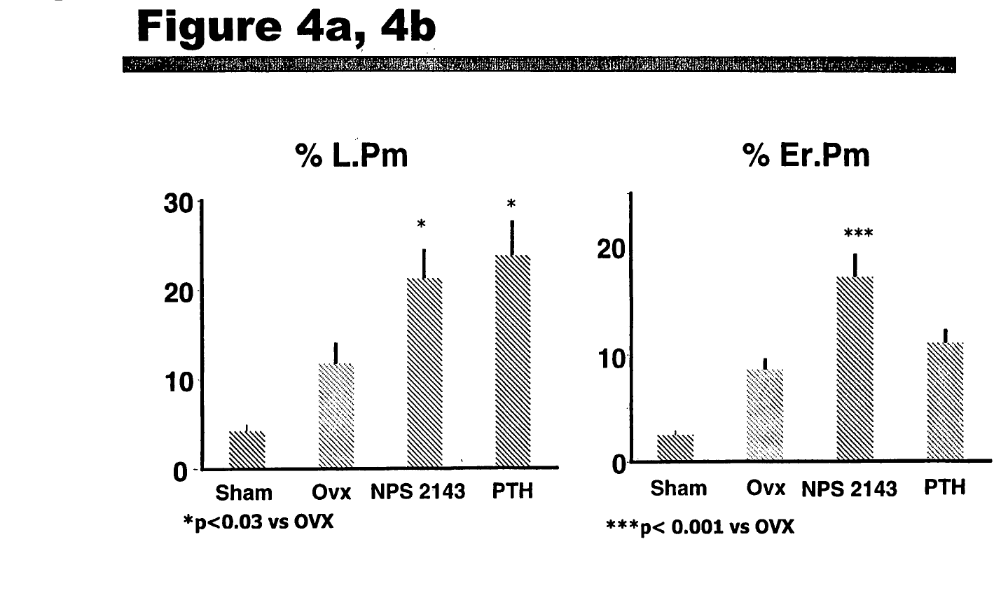

FIG. 3 represents circulating levels of calcilytic following administration of calcilytic according to Study 1.

Timed plasma samples were collected relative to administration of the compound as indicated.

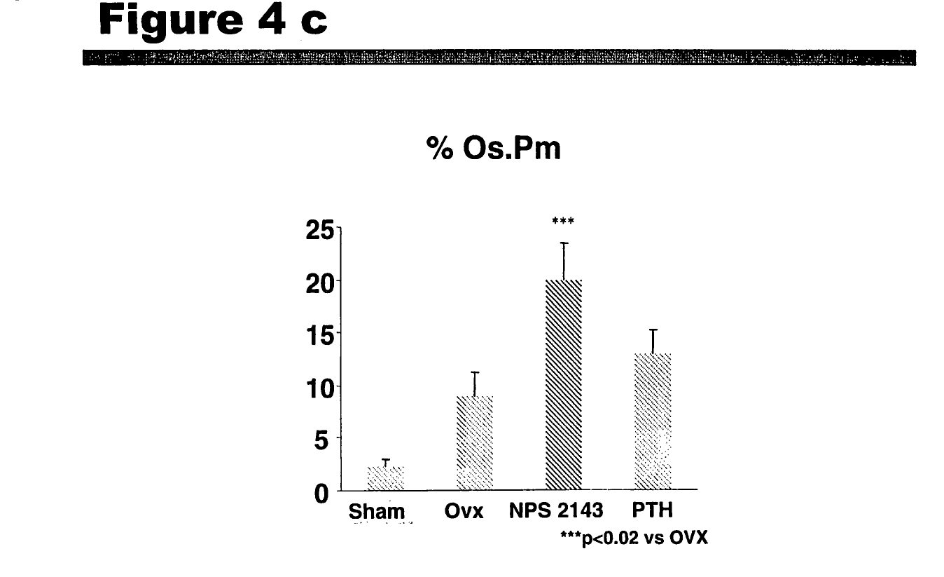

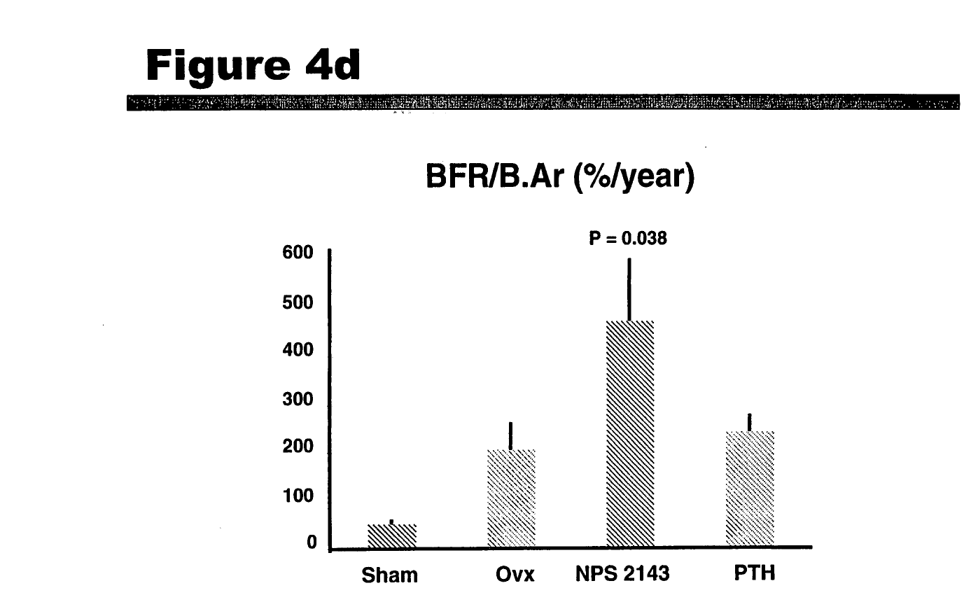

FIG. 4 represents dynamic histomorphometry on proximal tibiae from osteopenic rats following treatment with calcilytic or PTH according to Study 1.

FIG. 4a) represents % labeled perimeter (% L.Pm.)

FIG. 4b) represents % eroded surface (% Er.P)

FIG. 4c) represents % osteoid perimeter (% Os.Pm)

FIG. 4d) represents bone formation rate: bone area referent (BFR/B.Ar) %/year

Statistical significance is indicated: * P<0.05; **P<0.01

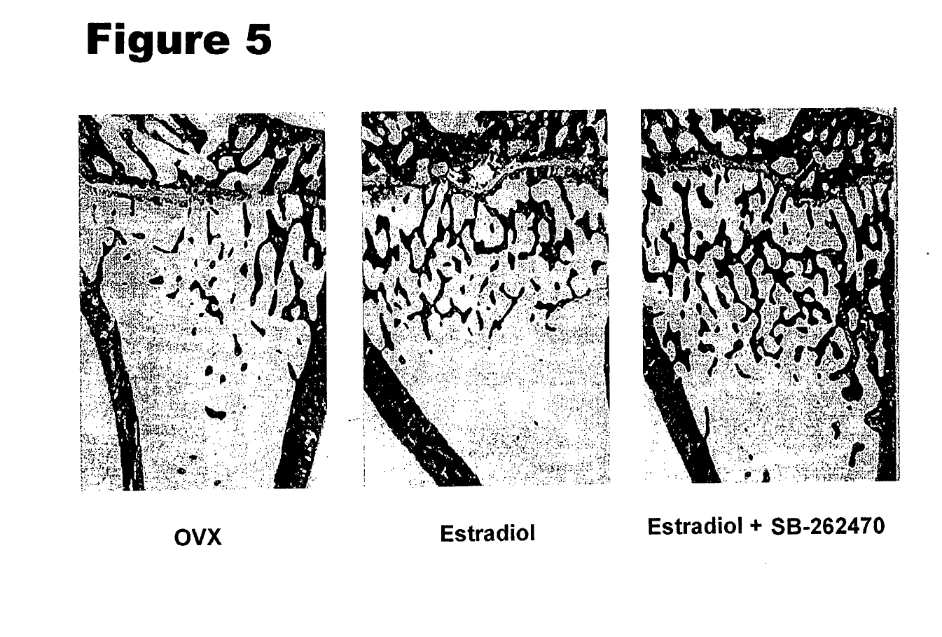

FIG. 5 represents sections of tibiae stained with Von Kossa from osteopenic ovx rats treated with estrogen+/−calcilytic according to Study 2.

Representative sections are shown from animals treated for the next 5 weeks with:

FIG. 5a) represents vehicle

FIG. 5b) represents 17β estradiol (s.c. pellet 0.01 mg/90 days)

FIG. 5c) represents NPS 2143 (100 umol/kg daily p.o.)

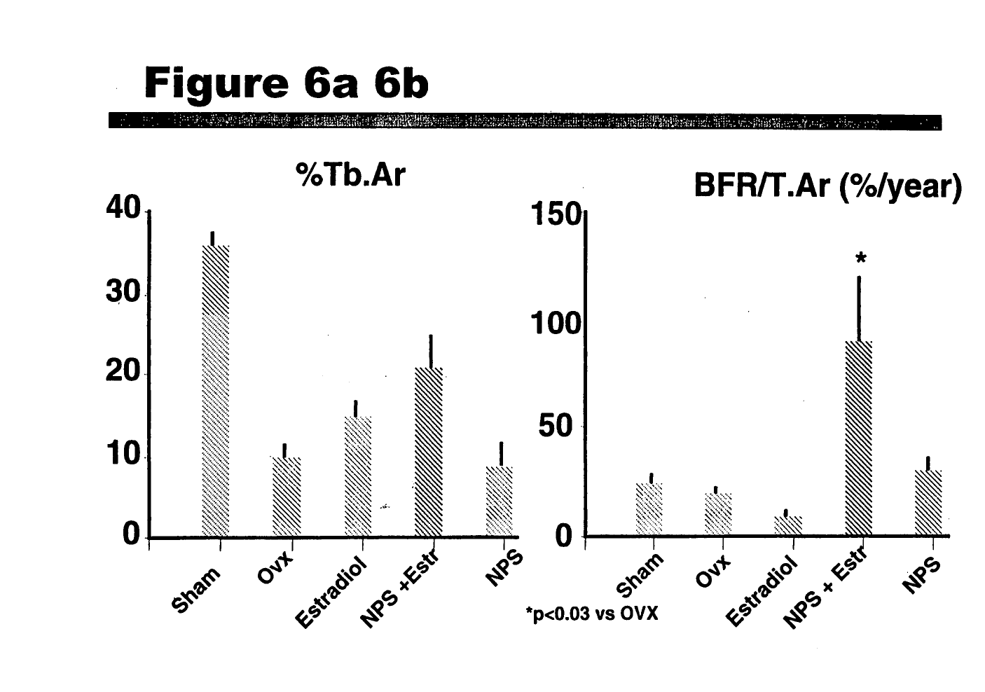

FIG. 6 represents histomorphometry on proximal tibiae from osteopenic rats following treatment with calcilytic plus/minus 17β estradiol according to Study 2.

FIG. 6a) represents % trabecular bone area (% Tb.Ar)

FIG. 6b) represents bone formation rate: tissue area referent (BFR/T.Ar) %/year

Statistical significance is indicated: * P<0.05; **P<0.01

### DETAILED DESCRIPTION OF THE INVENTION

The calcilytic compounds of the present invention include all calcilytic compounds. By “calcilytic compound”, it is meant that the compound is able to inhibit calcium receptor activity, thereby causing a decrease in one or more calcium receptor activities evoked by extracellular Ca²⁺. Such compounds include, but are not limited to a compound selected from the group consisting of:

N-[(2R-Hydroxy-3-[(3-chloro-2-cyano)phenoxy-propyl]-1,1-dimethyl-2-(2-naphthyl)ethyl amine hydrochloride;

N-[(2R-Hydroxy-3-[(3-chloro-2-cyano)phenoxy-propyl]-1,1-dimethyl-2-(4-methoxyphenyl)ethyl amine hydrochloride;

N-[(2R-Hydroxy-3-[(2,3-dichloro)phenoxy-propyl]-1,1-dimethyl-2-(4-methoxyphenyl)ethyl amine hydrochloride;

N-[(R)-2-Hydroxy-3-[2-cyano-4-[N-methyl-N-[3-carboxyphenyl)sulfonyl]amino]phenoxy]propyl]-1,1-dimethyl-2-(6-(1,2,3,4-tetrahydronaphthyl)ethylamine;

N-[(R)-2-Hydroxy-3-[2-cyano4-[N-methyl-N-[3-carboxyphenyl)sulfonyl]amino]phenoxy]propyl]-1,1-dimethyl-2-(Benzothien-3-yl)-ethylamine;

N-[(R)-2-Hydroxy-3-[2-cyano-4-[N-methyl-N-[3-carboxyphenyl)sulfonyl]amino]phenoxy]propyl]-1,1-dimethyl-2-(Benzothien-2-yl)-ethylamine;

N-[(R)-2-Hydroxy-3-[2-cyano-4-[N-methyl-N-[3-carboxyphenyl)sulfonyl]amino]phenoxy]propyl]-1,1-dimethyl-2-(decahydronapthalen-2-yl)ethylamine;

N-[(R)-2-Hydroxy-3-[2-cyano-4-[N-methyl-N-[3-carboxyphenyl)sulfonyl]amino]phenoxy]propyl]-1,1-dimethyl-4-phenylbutylamine;

N-[(R)-2-Hydroxy-3-[2-cyano-4-[N-methyl-N-[3-carboxyphenyl)sulfonyl]amino]phenoxy]propyl]-1,1-dimethyl-4-(2-methoxyphenyl)butylamine;

N-[2R-Hydroxy-3-[[2-cyano-4-N-methyl-N-[4-ethylcarboxyphenyl]sulfonyl]amino]phenoxy]propyl]-1,1-dimethyl-2-(2-napthyl)ethylamine;

N-[2R-Hydroxy-3-[[2-cyano-4-[N-methyl-N-[3-methylcarboxymethoxyphenyl]sulfonyl]amino]phenoxy]propyl]-1,1-dimethyl-2-(2-napthyl)ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-[2-napthyl]ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(1,2,3,4-tetrahydronaphth-6-yl)ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(benzothien-3-yl)-ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(benzothien-2-yl)-ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(decahydronapthalen-2-yl)ethylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-4-(2-methoxyphenyl)butylamine;

N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-4-phenylbutylamine;

N-[2R-Hydroxy-3-[[2-cyano-4-[N-benzyl-N-[4-methylphenyl]sulfonyl]amino]phenoxy]propyl]-1,1-dimethyl-2-[4-methoxyphenyl]ethylamine;

N-[2R-Hydroxy-3-[[2-cyano-4-[N-[4-benzyl]sulfonyl]amino]phenoxy]propyl]-1,1-dimethyl-2-[2-napthyl]ethylamine;

N-[2R-Hydroxy-3-[[2-cyano-5-[[4-carboxy]phenyl]phenoxy]propyl]-1,1-dimethyl-2-[napthyl]ethylamine;

N-[2R-Hydroxy-3-[[2-cyano-4-[[N-methyl-N-[3-carboxyl]phenyl]sulfonyl]amino]phenoxy]propyl]-1,1-dimethyl-2-[2-napthyl]ethylamine; N-[2R-Hydroxy-3-[[2-cyano-4-[[N-methyl-N-[3-methylcarboxyl]phenyl]sulfonyl]amino]phenoxy]propyl]-1,1-dimethyl-2-[2-napthyl]ethylamine;

N-[2R-Hydroxy-3-[[2-cyano-4-(2-phenyl-2-R,S-carboxyl)phenoxy]-propyl]-1,1-dimethyl-2-(2-naphthyl)ethylamine;

N-[2R-Hydroxy-3-[[2-cyano-4-(3-carboxypropyl)phenoxy]-propyl]-1,1-dimethyl-2-naphthylethylamine;

(N-[2R-Hydroxy-3-[[2-cyano-5-(3-carboxypropyl)phenoxy]-propyl]-1,1-dimethyl-2-naphthylethylamine; and

(N-[2R-Hydroxy-3-[2-[2-[6-aminomethyl]pyridyl]ethyloxy]-1,1-dimethyl-2-naphthylethylamine.

Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement.

Bone resorption is carried out by osteoclasts, which are multinuclear cells of hematopoietic lineage. The osteoclasts adhere to the bone surface and form a tight sealing zone, followed by extensive membrane ruffling on their apical (i.e., resorbing) surface. This creates an enclosed extracellular compartment on the bone surface that is acidified by proton pumps in the ruffled membrane, and into which the osteoclast secretes proteolytic enzymes. The low pH of the compartment dissolves hydroxyapatite crystals at the bone surface, while the proteolytic enzymes digest the protein matrix. In this way, a resorption lacuna, or pit, is formed. At the end of this phase of the cycle, osteoblasts lay down a new protein matrix that is subsequently mineralized. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

As used herein “anti-resorptive” means an agent capable of preventing, delaying or retarding bone resorption. Anti resorptive agents useful in the present invention include, but are not limited to, estrogen, 1, 25 (OH)₂ vitamin D3, calcitonin, bisphosphonate and cathepsin K inhibitors.

The present compounds can also be formulated as pharmaceutically acceptable salts and complexes thereof. Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfarnic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

In order to use a compound of the present invention or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdernal routes contains suitably from 0.01 mg to 100 mg/Kg, of a compound of Formula (I). A topical formulation contains suitably 0.01 to 5.0% of a compound of Formula (I). The active ingredient may be administered from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid (“PTH”) levels in a non-pulsatile manner. Increasing serum PTH levels may be helpful in treating diseases such as hypoparathyroidism, osteosarcoma, periodontal disease, fracture, osteoarthritis, rheumatoid arthritis, Paget's disease and osteoporosis.

The normal range for intact PTH in humans is about 10 to about 65 pg/ml. Increasing serum PTH may also be useful to prophylactically retard or prevent the onset of a disease. Prophylactic treatment can be performed, for example, on a person with a low serum PTH, or a person without low serum PTH, but where increasing PTH has a beneficial compensating effect. Preferably, the patient has an abnormally low serum PTH. As used herein, “abnormally low serum PTH” means a serum PTH level lower than that occurring in the general population, and is preferably an amount associated with a disease or onset of a disease.

Increasing serum PTH levels can be used to treat various diseases including bone and mineral related diseases.

Preferably, the duration of PTH level increase is 12 hours or longer, more preferably 18 hours or longer and most preferably 24 hours or longer.

Preferably, the increase in PTH is 3 fold or lower than the normal range for intact PTH in humans. More preferably, the increase in PTH level is 2-fold or lower than the normal range.

The present invention also provides compositions comprising the present compounds and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil. olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of the present invention or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

Biological Assays:

The following assays were performed.

Ovariectomized Rat Studies

Study 1

Seven month old virgin Sprague Dawley female rats were subjected to bilateral ovariectomy or sham surgery and the animals then held for a period of three months to allow the development of osteopenia. At that time a single sham group (n=10) and three groups of ovariectomized (ovx) animals (n=10-14) were assigned. The ovx groups were selected such that there was no significant difference in bone mineral density (“BMD”) of the lumbar spine, proximal tibia or distal femur between groups. Groups consisted of sham and ovx controls treated with dose vehicle (20% aqueous encapsin) and ovx groups treated with either N-[(2R-Hydroxy-3-[(3-chloro-2-cyano)phenoxy-propyl]-1,1-dimethyl-2-(2-naphthyl)ethyl amine hydrochloride (“calcilytic administered”) (100 umol/kg body weight daily p.o.) or rat PTH 1-34 (5 ug/kg body weight daily s.c.).

During the study blood samples were drawn for determination of circulating PTH and osteocalcin. BMD was determined by DXA (QDR-4500 Hologic, Waltham, Mass.) prior to treatment and at weeks 4 and 8. At term tibiae were removed for histological analysis. All animals received tetracycline 10 and 3 days prior to the start of dosing and calcein (10 mg/kg) 10 and 3 days prior to sacrifice.

Study 2

Animals were prepared and monitored as described above. Groups consisted of sham and ovx controls which were treated with oral dose vehicle (20% aqueous encapsin), and 4 additional ovx groups that received either N-[(2R-Hydroxy-3-[(3-chloro-2-cyano)phenoxy-propyl]-1,1-dimethyl-2-(2-naphthyl)ethyl amine hydrochloride (100 umol/kg/d, p.o.), 17β estradiol (s.c. pellet 0.01 mg/90 days), or N-[(2R-Hydroxy-3-[(3-chloro-2-cyano)phenoxy-propyl]-1,1-dimethyl-2-(2-naphthyl)ethyl amine hydrochloride+estradiol (each as above). Dosing continued for 5 weeks at which point the animals were sacrificed and the tibiae were collected for histological analysis.

Measurement of Circulating Compound and PTH Levels

Timed plasma samples were collected relative to administration of compound or PTH. PTH 1-34 was measured by RIA (Nichols Institute Diagnostics, San Juan Capistrano, Calif.). The concentrations of compound in the plasma were quantified by LC/MS/MS (limit of detection=10 ng/ml).

Histomorphometric Evaluation

Bone samples were dehydrated through increasing concentrations of ethanol, defatted in acetone and embedded in methyl methacrylate (Polysciences, Inc., Warrington, Pa.). Longitudinal undecalcified sections (5 μm) sections of the proximal tibial were cut on a Leica microtome (SM2500S); the tissue blocks had been prestained with Villanueva stain. Histomorphometric analysis was carried out using an Osteomeasure system (OsteoMetrics Incorpotated), without knowledge of group allocation. Measurements within the tibial metaphysis were restricted to a mean tissue area of approximately 8 mm² beginning 1 mm below the growth plate. Primary measurements included area of bone and marrow (mm²), bone area (mm²), perimeter of bone (mm), single and double-labelled perimeter (sL.Pm, dL.Pm, mm), osteoid perimeter (O.Pm, mm) and eroded perimeter (Er.P, mm). Derived indices included trabecular bone volume (% Tb.Ar), trabecular number (Th.N, mm⁻¹), trabecular thickness (Th.Th, μm), trabecular separation (Th.Sp, μm), bone formation rate, surface referent (BFR/Tb.Pm, μm³/μm²/year), BFR/Tb.Ar (bone area referent, %/year), BFR/B.Ar (tissue referent, %/year) and mineral apposition rate (MAR, μm/day), and percent labelled perimeter (% Lp). Statistical analysis was assessed by a two sided t-test.

Human Osteoclast-Mediated Bone Resorption Assay

The isolation of disaggregated human osteoclasts from fresh osteoclastoma tissue and the in vitro human osteoclast resorption assay were performed according to James, J. Bone Min. Res., Vol. 11, pp. 1453-1460. Briefly, human osteoclasts were seeded onto bovine cortical bone particles with compound or vehicle for 24 hours at 37° C. The culture media were then removed and the levels of the carboxy-terminal peptide of the α1 chain of human type I collagen were quantified as a biochemical readout of resorption, using a competitive binding enzyme linked immunosorbant assay (ELISA) (19) (Osteometer A/S, Rodovre, Denmark). The results are expressed as percent inhibition of resorption compared to supernatants derived from osteoclasts cultured in vehicle without inhibitor. IC₅₀ values are determined from the resultant dose response curves.

Fetal Rat Long Bone Resorption Assay

The assay was performed essentially as in Votta, Bone, Vol. 15, pp. 533-538, (1994). Timed-pregnant Sprague-Dawley rats (Taconic Farms, Germantown, N.Y.) were injected subcutaneously with 200 microcuries of ⁴⁵CaCl₂ on day 18 of gestation, housed overnight, then anesthetized with Innovar-Vet (Pittman-Moore, Mundelein, Ill.) and sacrificed by cervical dislocation. The fetuses were removed aseptically and the radii and ulnae were dissected free of surrounding soft tissue and cartilagenous ends. The bone rudiments (n=4) were subsequently cultured for 18-24 hours in BGJ_{b} medium (Sigma, St. Louis, Mo.) containing 1 mg/ml BSA, then transferred to fresh medium and cultured for an additional 48 hours in the absence or presence of PTH (human parathyroid hormone [1-34], Bachem, Torrence, Calif.) and the desired inhibitor. ⁴⁵Ca released into the medium and the residual ⁴⁵Ca in the bones (following solubilization in 5% TCA for 1 hour at room temperature) were quantitated by liquid scintillation spectrometry. Data are expressed as the percent ⁴⁵Ca released from treated bones as compared with corresponding control bones. Statistical differences were assessed by a one way analysis of variance (ANOVA). IC-50 values were based on data from two independent experiments.

Osteoblast cAMP Production and Alkaline Phosphatase Activity

cAMP accumulation was measured in both human TF274 osteoblastic cells (derived by immortalization of human bone marrow stromal cells) see James, supra, and primary human osteoblasts derived from explants of trabecular bone as described in Beresford, Biochim. Biophys. Acta, Vol. 801, pp. 58-65 (19884). cAMP levels in cell samples were measured using a non-radioactive protocol (Amersham kit). Alkaline phosphatase activity was determined using the standard colorimetric method as described previously in Gowen, Arth. Rheum., Vol. 31, pp. 1500-1507 (1988). N-[(2R-Hydroxy-3-[(3-chloro-2-cyano)phenoxy-propyl]-1,1-dimethyl-2-(2-naphthyl)ethyl amine hydrochloride was tested at 0.1, 1 and 10 uM. PTH 40 ng/ml was used as a positive control.

Results obtained from the assays described in Study 1 indicate that small but sustained elevation of PTH levels causes increased bone turnover with no net bone gain or loss.

Bone mineral density (BMD) was measured in vivo in the lumbar spine, distal femur and proximal tibia immediately before treatment and following eight weeks of dosing. Animals which had been ovariectomized three months previously had lost significant bone mass at all three skeletal sites: 15% at lumbar spine and proximal tibia, 24% at the distal femur. During the course of treatment bone mass was unaffected by treatment with N-[(2R-Hydroxy-3-[(3-chloro-2-cyano)phenoxy-propyl]-1,1-dimethyl-2-(2-naphthyl)ethyl amine, but was returned to pre-ovx levels after eight weeks of treatment with 5 ug/kg daily PTH in the proximal tibia (FIG. 1). Measurement of plasma PTH levels at the end of this experiment showed that the animals which received N-[(2R-Hydroxy-3-[(3-chloro-2-cyano)phenoxy-propyl]-1,1-dimethyl-2-(2-naphthyl)ethyl amine had elevated PTH levels (>100 pg/ml) which remained high at four hours after administration of the drug (FIG. 2). It is not known how long this elevation was sustained, although PTH levels were back to baseline after 24 hours (immediately prior to next dose). PTH levels in animals given 5 ug/kg PTH were in the same range as those dosed with N-[(2R-Hydroxy-3-[(3-chloro-2-cyano)phenoxy-propyl]-1,1-dimethyl-2-(2-naphthyl)ethyl amine, but were returned to baseline by 2-4 hours after dosing (FIG. 2). The differences in the duration of the PTH response can be explained by sustained exposure to N-[(2R-Hydroxy-3-[(3-chloro-2-cyano)phenoxy-propyl]-1,1-dimethyl-2-(2-naphthyl)ethyl amine, which was found to be elevated for up to eight hours (FIG. 3).

The difference in the PTH profile obtained under these two dosing conditions has allowed us to determine directly the effect of time of exposure to PTH on bone turnover. Dynamic histomorphometry of the proximal tibia showed that bone formation (% L.Pm., % Os.Pm) was elevated above the ovx control level by both PTH and N-[(2R-Hydroxy-3-[(3-chloro-2-cyano)phenoxy-propyl]-1,1-dimethyl-2-(2-naphthyl)ethyl amine, (FIGS. 4a, b). Mineral apposition rate was unchanged by any treatment. However bone resorption, as measured by % eroded perimeter, was significantly higher in the N-[(2R-Hydroxy-3-[(3-chloro-2-cyano)phenoxy-propyl]-1,1-dimethyl-2-(2-naphthyl)ethyl amine, group than in the PTH or ovx control groups (FIG. 4c). This is exemplified further by the dramatic increase in bone turnover demonstrated by the BFR/B.Ar. in the N-[(2R-Hydroxy-3-[(3-chloro-2-cyano)phenoxy-propyl]-1,1-dimethyl-2-(2-naphthyl)ethyl amine-treated compared with the other two groups (FIG. 4d). Thus the modest but prolonged elevation of PTH achieved by administration of calcilytic administered resulted in a dramatic increase in both bone formation and resorption, with no net bone gain or loss. PTH administered exogenously also increased both resorption and formation, but formation exceeded resorption, resulting in increased bone mass.

Results from Study 2 indicate that small but sustained elevation of PTH levels in the presence of an anti-resorptive agent causes increased bone turnover with net bone gain.

A second study was performed in which N-[(2R-Hydroxy-3-[(3-chloro-2-cyano)phenoxy-propyl]-1,1-dimethyl-2-(2-naphthyl)ethyl amine hydrochloride was administered daily for 4 weeks, in the presence or absence of estrogen to seven month old rats which had been ovx three months earlier. FIG. 5 shows representative sections of tibiae stained with Von Kossa from animals with no treatment following ovx (5a), treated with estrogen alone (5b) or treated with estrogen plus N-[(2R-Hydroxy-3-[(3-chloro-2-cyano)phenoxy-propyl]-1,1-dimethyl-2-(2-naphthyl)ethyl amine (5c). It is clear that co-administration of N-[(2R-Hydroxy-3-[(3-chloro-2-cyano)phenoxy-propyl]-1,1-dimethyl-2-(2-naphthyl)ethyl amine and estrogen (5c) caused increased bone mass over and above estrogen alone. Static histomorphometry of the proximal tibia (Table 1) showed that % trabecular area (% Tb.Ar.) was 72% lower in ovx animals compared to sham (P<0.0001). This bone loss was not significantly restored by estradiol. N-[(2R-Hydroxy-3-[(3-chloro-2-cyano)phenoxy-propyl]-1,1-dimethyl-2-(2-naphthyl)ethyl amine alone had no effect on the ovx-induced osteopenia. However, N-[(2R-Hydroxy-3-[(3-chloro-2-cyano)phenoxy-propyl]-1,1-dimethyl-2-(2-naphthyl)ethyl amine plus estrogen resulted in a two-fold increase in % Tb.Ar. over the ovx group (FIG. 6a). This appears to be due to an increase in trabecular thickness induced by N-[(2R-Hydroxy-3-[(3-chloro-2-cyano)phenoxy-propyl]-1,1-dimethyl-2-(2-naphthyl)ethyl amine plus estrogen (Table 1). The bone formation rate/tissue area was significantly elevated in the N-[(2R-Hydroxy-3-[(3-chloro-2-cyano)phenoxy-propyl]-1,1-dimethyl-2-(2-naphthyl)ethyl amine plus estrogen group (FIG. 6b). Elevation of bone formation rate/tissue area shows that bone mass is increasing in the area measured and reflects new bone formation on bone surfaces that are not being remodeled (modeling), a classic feature of PTH action. This appears to be a result of a decrease in resorption (presumably due to concurrent estrogen treatment) relative to the N-[(2R-Hydroxy-3-[(3-chloro-2-cyano)phenoxy-propyl]-1,1-dimethyl-2-(2-naphthyl)ethyl amine-treated animals in the face of maintained elevation of bone formation.

Direct Effects of N-[(2R-Hydroxy-3-[(3-chloro-2-cyano)phenoxy-propyl]-1,1-dimethyl-2-(2-naphthyl)ethyl Amine on Osteoblasts and Osteoclasts In Vitro

Since Ca²⁺ sensing receptors have been demonstrated on both osteoblasts and osteoclasts we studied the direct effects of N-[(2R-Hydroxy-3-[(3-chloro-2-cyano)phenoxy-propyl]-1,1-dimethyl-2-(2-naphthyl)ethyl amine on both osteoblasts and osteoclasts in vitro were studied.

Osteoblast Activity

While PTH caused a two-fold increase in cAMP levels in both cell types used, N-[(2R-Hydroxy-3-[(3-chloro-2-cyano)phenoxy-propyl]-1,1-dimethyl-2-(2-naphthyl)ethyl amine—had no effect on basal or PTH-induced cAMP levels. Treatment of TF274 cells with N-[(2R-Hydroxy-3-[(3-chloro-2-cyano)phenoxy-propyl]-1,1-dimethyl-2-(2-naphthyl)ethyl amine did not result in any change in alkaline phosphatase activity, nor was PTH-induced alkaline phosphatase affected by N-[(2R-Hydroxy-3-[(3-chloro-2-cyano)phenoxy-propyl]-1,1-dimethyl-2-(2-naphthyl)ethyl amine. N-[(2R-Hydroxy-3-[(3-chloro-2-cyano)phenoxy-propyl]-1,1-dimethyl-2-(2-naphthyl)ethyl amine demonstrated some toxicity in vitro at the 10 uM concentration.

Osteoclast Activity

N-[(2R-Hydroxy-3-[(3-chloro-2-cyano)phenoxy-propyl]-1,1-dimethyl-2-(2-naphthyl)ethyl amine had no effect on human osteoclast mediated bone resorption at concentrations up to 3 uM, while an inhibitor of cathepsin K, 3,11-bis (2-methylpropyl)4,7,10-trioxo-2,5,6,8,9,12-hexaazatridecanedioate inhibited with an IC₅₀ of 0.9 uM. This assay is limited by its sensitivity to DMSO, so concentrations of N-[(2R-Hydroxy-3-[(3-chloro-2-cyano)phenoxy-propyl]-1,1-dimethyl-2-(2-naphthyl)ethyl amine above 3 uM could not be tested. In the fetal rat long bone assay N-[(2R-Hydroxy-3-[(3-chloro-2-cyano)phenoxy-propyl]-1,1-dimethyl-2-(2-naphthyl)ethyl amine inhibited resorption with an IC₅₀ of 11.3+/−3 uM. The mechanism of this inhibition is not understood and since the effect occurs at concentrations approximately 300 fold higher than the IC50 for Ca²⁺ receptor-mediated Ca²⁺ mobilization it may well be unrelated to any effect on the Ca²⁺ receptor. The possibility that it could be related to toxicity cannot be ruled out.

The above experiments demonstrated that a small orally active compound can be designed which induces endogenous PTH secretion sufficiently to stimulate bone turnover. The pharmacokinetic characteristics of this molecule are such that a prolonged elevation of PTH (>4 hours) is obtained. This has allowed us to examine the role of the duration of PTH elevation at low levels of circulating PTH. When PTH was elevated for greater than four hours bone turnover was further elevated, but remained in balance leading to no net loss or gain. The co-therapy experiment was performed with estrogen and antagonist co-treatment resulting in an increase in bone formation as measured histomorphometrically.

It is well known that chronic elevation of PTH, such as that seen in hyperparathyroidism, leads to bone loss and abnormal bone histology. Dobnig et al., supra, showed that subcutaneous infusion of high doses of PTH (40 and 80 ug/kg/day) over periods of 2 hours or more led to rapid loss in body weight, hypercalcemia and histological abnormalities in the skeleton consistent with changes seen in hyperparathyroidism. In our study the much smaller increases in PTH, although sustained, did not lead to these adverse effects. However, the anabolic effect of PTH was still lost with sustained exposure. This suggests that a mild hyperparathyroid condition, whether natural or pharmacologically-induced, could be asymptomatic.

The present data also demonstrate that, with regard to the amount of PTH secreted in response N-[(2R-Hydroxy-3-[(3-chloro-2-cyano)phenoxy-propyl]-1,1-dimethyl-2-(2-naphthyl)ethyl amine, a low duration and low fold increase in PTH levels had profound effects on bone turnover. Most published studies on effects of PTH in rats have used a dose of 80 ug/kg. This dose leads to a circulating level of approximately 5,000-14,000 pg/ml, compared to the 150-200 pg/ml in our studies. This demonstrates that very low doses of PTH effectively modulate bone turnover. This is also illustrated by the much lower doses used in the clinical studies performed recently in which approximate doses of 0.4-0.8 ug/kg body weight led to increased bone mass (8,24). 0.4 ug/kg led to circulating levels of approximately 90 pmol/l of PTH 1-34 at 30 minutes after dosing (25). This is an approximately three-fold increase in circulating PTH levels. Thus it appears that the PTH stored in the parathyroid gland will be sufficient to cause an anabolic effect if released in response to a Ca²⁺ receptor antagonist.

The above data demonstrated for the first time that stimulation of endogenous parathyroid hormone secretion using an antagonist of the parathyroid cell Ca²⁺ receptor results in increased bone formation and resorption. In the presence of an anti-resorptive agent N-[(2R-Hydroxy-3-[(3-chloro-2-cyano)phenoxy-propyl]-1,1-dimethyl-2-(2-naphthyl)ethyl amine, caused an increase in bone mass. This provides the basis for the development of a novel class of anabolic agent for the treatment of osteoporosis.

All publications, including but not limited to patents and patent applications cited in this specification are herein incorporated by reference as if each individual publication were specifically and individually indicated to be incorporated by reference as though fully set forth.

What is claimed is:

1. A method of treating a disease or disorder characterized by an abnormal bone or mineral homeostasis which comprises administering to a subject in need of treatment thereof an effective amount of a calcilytic compound in conjunction with an effective amount of an anti-resorptive agent.

2. A method according to claim 1 wherein the calcilytic compound is selected from the group consisting of:
N-[(2R-Hydroxy-3-[(3-chloro-2-cyano)phenoxy-propyl]-1,1-dimethyl-2-(2-naphthyl)ethyl amine hydrochloride;
N-[(2R-Hydroxy-3-[(3-chloro-2-cyano)phenoxy-propyl]-1,1-dimethyl-2-(4-methoxyphenyl)ethyl amine hydrochloride;
N-[(2R-Hydroxy-3-[(2,3-dichloro)phenoxy-propyl]-1,1-dimethyl-2-(4-methoxyphenylmethyl amine hydrochloride;
N-[(R)-2-Hydroxy-3-[2-cyano-4-[N-methyl-N-[3-carboxyphenyl)sulfonyl]amino]phenoxy]propyl]-1,1-dimethyl-2-(6-(1,2,3,4-tetrahydronaphthyl)ethylamine;
N-[(R)-2-Hydroxy-3-[2-cyano-4-[N-methyl-N-[3-carboxyphenyl)sulfonyl]amino]phenoxy]propyl]-1,1-dimethyl-2-(Benzothien-3-yl)-ethylamine;
N-[(R)-2-Hydroxy-3-[2-cyano-4-[N-methyl-N-[3-carboxyphenyl)sulfonyl]amino]phenoxy]propyl]-1,1-dimethyl-2-(Benzothien-2-yl)-ethylamine;
N-[(R)-2-Hydroxy-3-[2-cyano-4-[N-methyl-N-[3-carboxyphenyl)sulfonyl]amino]phenoxy]propyl]-1,1-dimethyl-2-(decahydronapthalen-2-yl)ethylamine;
N-[(R)-2-Hydroxy-3-[2-cyano-4-[N-methyl-N-[3-carboxyphenyl)sulfonyl]amino]phenoxy]propyl]-1,1-dimethyl-4-phenylbutylamine;
N-[(R)-2-Hydroxy-3-[2-cyano-4-[N-methyl-N-[3-carboxyphenyl)sulfonyl]amino]phenoxy]propyl]-1,1-dimethyl-4-(2-methoxyphenyl)butylamine;
N-[2R-Hydroxy-3-[[2-cyano-4-[N-methyl-N-[4-ethylcarboxyphenyl]sulfonyl]amino]phenoxy]propyl]-1,1-dimethyl-2-(2-napthyl)ethylamine;
N-[2R-Hydroxy-3-[[2-cyano-4-[N-methyl-N-[3-methylcarboxymethoxyphenyl]sulfonyl]amino]phenoxy]propyl]-1,1-dimethyl-2-(2-napthyl)ethylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-[2-napthyl]ethylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(1,2,3,4-tetrahydronaphth-6-yl)ethylamine.
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(benzothien-3-yl)-ethylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(benzothien-2-yl)-ethylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-2-(decahydronapthalen-2-yl)-ethylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-4-(2-methoxyphenyl)butylamine;
N-[2R-Hydroxy-3-[[2-cyano-4-[[N-methylsulfonyl]-N-[[[1-[2-[6-methyl]amino]pyridyl]ethyl]amino]phenoxy]propyl]-1,1-dimethyl-4-phenylbutylamine;
N-[2R-Hydroxy-3-[[2-cyano-4-[N-benzyl-N-[4-methylphenyl]sulfonyl]amino]phenoxy]propyl]-1,1-dimethyl-2-[4-methoxyphenyl]ethylamine;
N-[2R-Hydroxy-3-[[2-cyano-4-[N-[4-benzyl]sulfonyl]amino]phenoxy]propyl]-1,1-dimethyl-2-[2-napthyl]ethylamine;
N-[2R-Hydroxy-3-[[2-cyano-5-[[4-carboxy]phenyl]phenoxy]propyl]-1,1-dimethyl-2-[napthyl]ethylamine;
N-[2R-Hydroxy-3-[[2-cyano-4-[[N-methyl-N-[3-carboxyl]phenyl]sulfonyl]amino]phenoxy]propyl]-1,1-dimethyl-2-[2-napthyl]ethylamine; N-[2R-Hydroxy-3-[[2-cyano-4-[[N-methyl-N-[3-methylcarboxyl]phenyl]sulfonyl]amino]phenoxy]propyl]-1,1-dimethyl-2-[2-napthyl]ethylamine;
N-[2R-Hydroxy-3-[[2-cyano-4-(2-phenyl-2-R,S-carboxyl)phenoxy]-propyl]-1,1-dimethyl-2-(2-naphthyl)ethylamine;
N-[2R-Hydroxy-3-[[2-cyano-4-(3-carboxypropyl)phenoxy]-propyl]-1,1-dimethyl-2-naphthylethylamine;
(N-[2R-Hydroxy-3-[[2-cyano-5-(3-carboxypropyl)phenoxy]-propyl]-1,1-dimethyl-2-naphthylethylamine; and
(N-[2R-Hydroxy-3-[2-[2-[6-aminomethyl]pyridyl]ethyloxy]-1,1-dimethyl-2-naphthylethylamine.

3. A method according to claim 2 wherein the anti-resorptive agent is selected from the group consisting of: estrogen, 1, 25 (OH)₂ vitamin D3, calcitonin, selective estrogen receptor modulators, vitronectin receptor antagonists, V-H+-ATPase inhibitors, src SH2 antagonists, bisphosphonates and cathepsin K inhibitors.

4. A method according to claim 1 wherein the bone or mineral disease or disorder is selected from the group consisting of: periodontal disease, fracture healing, osteoarthritis, rheumatoid arthritis, Paget's disease, humoral hypercalcemia of malignancy, metastatic bone disease, joint replacement and osteoporosis.

5. A method according to claim 3 wherein the bone or mineral disease or disorder is osteoporosis.

6. A method according to claim 1 wherein the calcilytic agent causes an increase in serum PTH levels of 3-fold or higher.

7. A method according to claim 1 wherein the calcilytic agent causes an increase in serum PTH levels of 2-fold or higher.

8. A method of treating a disease or disorder characterized by an abnormal bone or mineral homestasis which comprises administering to a subject in need of treatment thereof an effective amount of an anabolic compound in conjunction with an effective amount of an anti-resorptive agent.
